(19) United States
(12) Patent Application Publication
(10) Pub. No.: US 2026/0053770 A1
(43) Pub. Date: Feb. 26, 2026

(54) COMPOSITION AND METHOD FOR TREATING ARTHRITIS

(71) Applicants: Binoy K. Bordoloi, Bridgewater, NJ (US); Ronen K. Bordoloi, Alameda, CA (US); Nicole Heeney, Alameda, CA (US); Deepa Mehta, Nainital (IN); KULWANT SINGH SAINI, GURUGRAM (IN); Debojit Saikia, Guwahati (IN); Sachin Kumar, North Guwahati (IN)

(72) Inventors: Binoy K. Bordoloi, Bridgewater, NJ (US); Ronen K. Bordoloi, Alameda, CA (US); Nicole Heeney, Alameda, CA (US); Deepa Mehta, Nainital (IN); KULWANT SINGH SAINI, GURUGRAM (IN); Debojit Saikia, Guwahati (IN); Sachin Kumar, North Guwahati (IN)

(73) Assignee: Bordoloi Biotech LLC, Bridgewater, NJ (US)

(21) Appl. No.: 18/813,748

(22) Filed: Aug. 23, 2024

(51) Int. Cl. A61K 31/343; A61K 31/11; A61K 31/216; A61K 36/758; A61K 36/899; A61K 36/906; A61P 29/00

(52) U.S. Cl. CPC A61K 31/343; A61K 31/11; A61K 31/216; A61K 36/758; A61K 36/899; A61K 36/906; A61P 29/00

(57) ABSTRACT

The present invention discloses a composition and method for treating arthritis. The composition comprises a binary blend of molecules including citral, methyl cinnamate, and epicurzerenone. The binary blend exhibits synergistic efficacy in modulating inflammatory cytokines. The citral, methyl cinnamate, and epicurzerenone, are derived from essential oils of Cymbopogon citratus, Hedychium spicatum, and Zanthoxylum armatum, respectively. Both individual and binary blend exhibits downregulation of pro-inflammatory cytokines IL-2, IL-6, and TNF-α, and upregulation of anti-inflammatory cytokine IL-10.

### TECHNICAL FIELD

The present invention generally relates to arthritis. More particularly, the present invention relates to a composition and method for treating arthritis.

### BACKGROUND

Rheumatoid arthritis (RA) is a chronic autoimmune disorder that affects individuals by causing pain, swelling and deformity in joints. Additionally, RA has a potential to cause chronic pain and irreversible damage to tendons, ligaments, and bones. Further, it might also cause damage to various body system including skin, eyes, lungs, heart, and blood vessels. In autoimmune RA, the immune system mistakenly identifies healthy body tissue cells as foreign invaders and releases inflammatory chemicals that attack the healthy cells. Here, the immune system primarily targets the lining of the joints called synovium that produces fluid to help the joint to move smoothly. The resulting inflammation causes the synovium to thicken, leading to painful swelling, stiffness, and potential loss of joint function over time. Initially, RA affects small peripheral joints that have an enormous impact on hand and foot functions, and as the condition progresses it may spread to larger joints.

Management of RA involves a combination of medication, lifestyle changes, and sometimes surgery to alleviate symptoms and improve quality of life. Medications commonly used include nonsteroidal anti-inflammatory drugs (NSAIDs) to reduce pain and inflammation, disease-modifying antirheumatic drugs (DMARDs) to slow disease progression, and biologic agents that target specific components of the immune system. In addition to medications, physical therapy and regular exercise are essential for maintaining joint flexibility and strength. In some cases, surgical interventions, such as joint replacement or tendon repair, may be necessary to restore function and alleviate pain. Further, a few related patent references are discussed as follows.

CN108186618 of Chengdu Medical College entitled “The new application of citral and its derivative in MRSA infectious disease medicaments are prepared” discloses a composition to treat methicillin-resistant Staphylococcus aureus (MRSA) infections. The composition comprises 0.01-0.99 part of citral and 0.01-0.99 part of beta-lactam antibiotics. Further, the citral and the beta lactam antibiotics are administered simultaneous or separately. The citral or the derivatives thereof are natural medicines. Further, the citral inhibits the growth of MRSA or kill the MRSA with a single use. The citral allows to relieve the oxidative stress and inflammatory reaction caused by MRSA infection. The beta-lactam antibiotics enhance the MRSA resistance activity, and has good synergistic effect.

WO2023147641 of Luiz Francisco Pianowski entitled “Anti-inflammatory, immunomodulatory and analgesic agents from tree exudates” discloses about an extract of resin from species of Boswellia and Commiphora. The species of Boswellia and Commiphora is used to treat chronic inflammation conditions, potentially leading to cancer, or autoimmune diseases. The autoimmune disease includes Alzheimer, Crohn's syndrome, psoriasis, and multiple sclerosis. Further, the selection of resin extracts or resin components that might modulate the activity of protein kinases includes PKC isoforms activity.

Although, the existing patent references disclose usage of citral and its derivatives for their antimicrobial and anti-inflammatory properties, these patent references lack to explore the potential applications for RA. Further, the existing references disclose compositions and extracts for other conditions and lack to address RA specifically.

Therefore, there is a need for a composition and method to treat arthritis (could be for both RA and osteoarthritis [OA]). The composition needs to incorporate active components that specifically address the autoimmune processes involved in this condition. In addition, the anti-inflammation response is not specific to autoimmune RA. This composition and treatment are applicable to any form of RA with inflammation.

### SUMMARY

The present invention discloses a composition for treating arthritis. The composition comprises a binary blend of molecules including citral, methyl cinnamate, and epicurzerenone. The binary blend exhibits synergistic efficacy in modulating inflammatory cytokines. The citral, methyl cinnamate, and epicurzerenone, are derived from essential oils of Cymbopogon citratus, Hedychium spicatum, and Zanthoxylum armatum, respectively. The binary blend exhibits downregulation of pro-inflammatory cytokines and upregulation of anti-inflammatory cytokines. In one embodiment, the binary blend exhibits downregulation of pro-inflammatory cytokines IL-2, IL-6, and TNF-α, and upregulation of anti-inflammatory cytokine IL-10.

In one embodiment, the binary blend comprises citral of about 2. 5 μM, methyl cinnamate of about 10 μM, and epicurzerenone of about 3 μM. In another embodiment, the binary blend comprises citral of about 2 μM, methyl cinnamate of about 10 μM, and epicurzerenone of about 3 μM. The binary blend is formulated in a pharmaceutically acceptable carrier for at least one of oral, injection and topical administration. The binary blend is administered at doses effective for alleviating symptoms of arthritis in a subject.

The present invention further discloses a method for treating arthritis. The method comprises a step of administering to a subject in need thereof, a therapeutically effective amount of a binary blend of molecules comprising citral, methyl cinnamate, and epicurzerenone. The method further comprises a step of formulating the binary blend in a pharmaceutically acceptable carrier for at least one of oral, injection and topical administration.

In one embodiment, the step of administering the binary blend involves selecting concentrations of citral, methyl cinnamate, and epicurzerenone of about 2.5 μM, 10 μM, and 3 μM, respectively. In another embodiment, the step of administering the binary blend involves selecting concentrations of citral, methyl cinnamate, and epicurzerenone of about 2 μM, 10 μM, and 3 μM, respectively. The administration of the binary blend results in a synergistic efficacy demonstrated by downregulation of pro-inflammatory cytokines and upregulation of anti-inflammatory cytokines compared to individual compounds. The administration of the binary blend alleviates symptoms of arthritis by downregulating pro-inflammatory cytokines IL-2, IL-6, and TNF-α, and upregulating anti-inflammatory cytokine IL-10, thereby providing therapeutic benefit to the subject.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1A is a graph showing the cytotoxicity of citral in A549 cells is determined based on the dose-response using MTT assay, according to an embodiment of the present invention.

FIG. 1B is a graph showing the cytotoxicity of citral in A549 cells by exposing A549 cells to different concentrations of citral for 48 hours using MTT assay, according to an embodiment of the present invention.

FIG. 2A is a graph showing the cytotoxicity of methyl cinnamate in A549 cells is determined based on the dose-response using MTT assay, according to an embodiment of the present invention.

FIG. 2B is a graph showing the cytotoxicity of methyl cinnamate in A549 cells, which is determined by exposing A549 cells to different concentrations of methyl cinnamate for 48 hours using MTT, according to an embodiment of the present invention.

FIG. 3 exemplarily illustrates microscopic white light images showing toxicity in A549 cell line after 48 hours of treatment with methyl cinnamate at different concentrations A) 0 μm (control) B) 12.5 μm C) 25 μm D) 50 μm E) 100 μm F) 200 μm G) 400 μm, according to an embodiment of the present invention.

FIG. 4 is a graph showing the cytotoxicity of epicurzerenone in A549 cells, according to an embodiment of the present invention.

FIG. 5 is a schematic showing Agarose gel (1%) electrophoresis of the total RNA isolated, according to an embodiment of the present invention.

FIG. 6 is a graph showing an anti-inflammatory effect of citral upon post-treatment with LPS induced A549 cells for 48 hours, according to an embodiment of the present invention.

FIG. 7 is a graph showing an anti-inflammatory effect of methyl cinnamate upon post-treatment with LPS induced A549 cells for 48 hours, according to an embodiment of the present invention.

FIG. 8 is a graph showing an anti-inflammatory effect of epicurzerenone upon post-treatment with LPS induced A549 cells for 48 hours, according to an embodiment of the present invention.

FIG. 9 is a graph showing an anti-inflammatory effect of the binary blend comprising 2.5 M of citral and 10 UM of methyl cinnamate post-treatment with LPS induced A549 cells, according to an embodiment of the present invention.

FIG. 10 is a graph showing an anti-inflammatory effect of the binary blend comprising 10 μM of methyl cinnamate and 3 μM of epicurzerenone post-treatment with LPS induced A549 cells, according to an embodiment of the present invention.

FIG. 11 is a graph showing an anti-inflammatory effect of the binary blend comprising 2.5 μM of citral and 3 μM of epicurzerenone post-treatment with LPS induced A549 cells, according to an embodiment of the present invention.

### DETAILED DESCRIPTION OF EXAMPLE EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

The present invention discloses a composition for treating arthritis. The composition comprises a binary blend of molecules including citral, methyl cinnamate, and epicurzerenone. Further, the binary blend exhibits synergistic efficacy in modulating inflammatory cytokines. Further, the citral, methyl cinnamate, and epicurzerenone, are derived from essential oils of Cymbopogon citratus, Hedychium spicatum, and Zanthoxylum armatum, respectively.

The binary blend exhibits downregulation of pro-inflammatory cytokines and upregulation of anti-inflammatory cytokines. The binary blend further exhibits downregulation of pro-inflammatory cytokines IL-2, IL-6, and TNF-α, and upregulation of anti-inflammatory cytokine IL-10. In one embodiment, the binary blend comprises citral of about 2. 5 μM, methyl cinnamate of about 10 μM, and epicurzerenone of about 3 μM. In another embodiment, the binary blend comprises citral of about 2 μM, methyl cinnamate of about 10 μM, and epicurzerenone of about 3 μM. The binary blend is formulated in a pharmaceutically acceptable carrier for at least one of oral, injection and topical administration. In one embodiment, the composition is formulated in tablet or capsule form. Further, the binary blend is administered at doses effective for alleviating symptoms of arthritis in a subject. In one embodiment, the active compounds were commercially obtained from Sigma (citral), Sigma (methyl cinnamate), and ChemFaces (epicurzerenone).

The present invention further discloses a method for treating arthritis. The method comprises a step of administering to a subject in need thereof, a therapeutically effective amount of a binary blend of molecules comprising citral, methyl cinnamate, and epicurzerenone. The method further comprises a step of formulating the binary blend in a pharmaceutically acceptable carrier for at least one of oral, injection and topical administration.

In one embodiment, the step of administering the binary blend involves selecting concentrations of citral, methyl cinnamate, and epicurzerenone of about 2.5 μM, 10 μM, and 3 μM, respectively. In another embodiment, the step of administering the binary blend involves selecting concentrations of citral, methyl cinnamate, and epicurzerenone of about 2 μM, 10 μM, and 3 μM, respectively.

The administration of the binary blend results in a synergistic efficacy demonstrated by the downregulation of pro-inflammatory cytokines and the upregulation of anti-inflammatory cytokines compared to individual compounds. The administration of the binary blend alleviates symptoms of arthritis by downregulating pro-inflammatory cytokines IL-2, IL-6, and TNF-α, and upregulating anti-inflammatory cytokine IL-10, thereby providing therapeutic benefit to the subject.

### EXAMPLES

Non-limiting examples of the invention will be further described in greater detail by reference to specific examples, which should not be construed as in any way limiting the scope of the invention.

The key molecules in each of the three essential oils are analyzed and in vitro studies of gene expression of the inflammatory markers at IIT-G are conducted. The three molecules are selected from the three essential oils as follows. The essential oils are lemongrass oil from Cymbopogon citratus, tumuru oil from Zanthoxylum armatum, and sati oil from Hedychium spicatum. The molecules are Citral (37.4% of the oil), Methyl Cinnamate (17.1% of the oil), and epicurzerenone (29.7% of the oil) from lemongrass oil, tumuru oil and sati oil, respectively.

The citral is also referred as A, methyl cinnamate is also referred as B, and epicurzerenone is also referred as C. The preliminary data of three molecules is from gene expression studies of inflammation demonstrated on binary blends, that includes citral and methyl cinnamate (A+B), methyl cinnamate and epicurzerenone (B+C), and epicurzerenone and citral (C+A), showed significantly higher efficacy than the corresponding individual molecules A, B or C at 1/10^{th} of the toxic dose (IC₅₀) level of their respective concentrations. Further, 1/10 of an in vitro dose is very conservative, and much higher doses could be given in vivo.

The present invention discloses in vitro studies on binary blends of molecules. The binary blend molecules include citral and methyl cinnamate (A+B), methyl cinnamate and epicurzerenone (B+C), and citral and epicurzerenone (C+A). The binary blend contains each molecule in most optimum concentration. The optimum concentration is about 1/10^{th} of IC₅₀ toxic dose of the respective compound in each of the three blends.

The present invention further includes in vivo study to check efficacy on the three binary blends of (A+B), (B+C) and (C+A) in an arthritic mice model at the desired dose based on 1/10^{th} IC₅₀ toxicity data of the respective individual molecules.

The present invention further conducts in vivo safety evaluation for a few selected parameters to set the in vivo efficacy doses. The parameters involve a single oral dose of three group and a vehicle control to groups of animals for 7 days. Further, the parameters include clinical observations, BW, mortality, and gross necropsy on major organs. Further, the parameter includes Low, mid, and high doses. The present invention study involves animals that include rabbits and rats. The study uses rabbits for subcutaneous irritation testing following ISO 10993-10 standards protocol for Intracutaneous Reactivity to assess for local irritancy. Further, the study uses rats for acute oral systemic toxicity testing (IC₅₀). LD50 precision is not necessary in an enteral model and the pharmacokinetics (PK) and toxicokinetics (TK) will be evaluated later.

Further, the anti-inflammatory response will be assessed by observing a downregulation of pro-inflammatory cytokines and an upregulation of anti-inflammatory cytokines. The pro-inflammatory cytokines promote the cause of inflammation and include interleukin-2 (IL-2), interleukin-6 (IL-6), and tumor necrosis factor-alpha (TNF-α), while the anti-inflammatory cytokines reduce inflammation and includes interleukin-10 (IL-10). To evaluate cytokine modulation various strategies are employed that includes ELISA kits to determine cytokine levels, anti-cytokine antibodies at the protein level, and primers to assess cytokine modulation at the RNA level. In present invention, the cytokine-specific primers toxicokinetic for both anti-inflammatory (IL-10) and pro-inflammatory cytokines (IL-2, IL-6, TNFα) are synthesized and used to study the cytokine modulation via real-time PCR. Further, the housekeeping gene β-actin is used to normalize the gene expression.

The in vitro experiment is carried out to check the toxicity of each active component. The invitro experiment is conducted in the laboratory under controlled conditions. Further, an available human lung cancer cell line A549 was used for in vitro experiment. A549 cell line are widely used to ensure reliable results in studies. Further, the study involved toxicity testing of each active component using a MTT (3-(4, 5-dimethylthiazolyl-2)-2, 5-diphenyltetrazolium bromide) assay. MTT assay, a colorimetric assay that measures cellular metabolic activity as an indicator of cell viability, proliferation, and cytotoxicity. Further, the MTT assay determines the concentration of a drug that causes 50% toxicity is known as an inhibitory concentration (IC₅₀) value. In the present invention, the concentration is at least 10 folds less than the IC₅₀ value, which showed a satisfactory response and chosen for the experiment to limit toxicity. Approximately, 50% of cytokine modulation was considered satisfactory for the results.

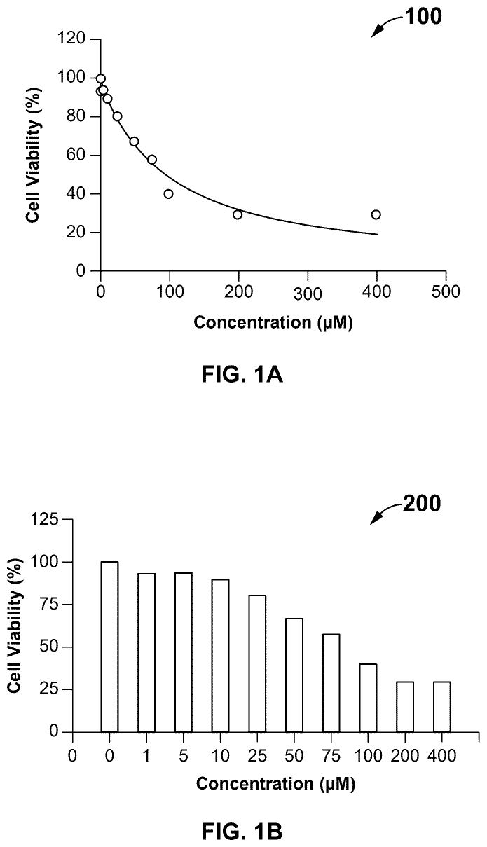

Further, the steps involved in the experiment to check the toxicity is conducted using the individual active ingredients. A549 is grown in a 96 wells ELISA plate. Once a monolayer is formed, treat the cells with different concentrations of the active component diluted in serum-free media. Further, incubate the 96 wells ELISA plate for 48 hours to 72 hours and observe the toxicity for every 24 hours. After 72 hours, the media is aspired and discarded. Further, add diluted MTT solution to the 96 wells ELISA plate and incubated for 3 hours at 37° C. After incubation, add dimethyl sulfoxide (DMSO) to the wells and measure the absorbance at 590 nm. Further, the results were interpreted by observing the intensity of the color. The intensity of the color is directly proportional to the cell viability. Further, for each active component, the IC₅₀ value was determined based on observations in FIG. 1A to FIG. 4.

FIG. 1A and FIG. 1B illustrates the observation of cytotoxicity assay of citral in A549 cells. FIG. 1A illustrates a graph 100 of cytotoxicity of citral in A549 cells, which is determined based on the dose-response using MTT assay. The 50% cytotoxic concentration (IC₅₀) was calculated for the compound using nonlinear regression analysis of GraphPad Prism software (version 8.0). FIG. 1B illustrates a graph 200 of the cytotoxicity of citral in A549 cells by exposing A549 cells to different concentrations of citral for 48 hours using MTT assay. Values are the mean±SE of three independent experiments.

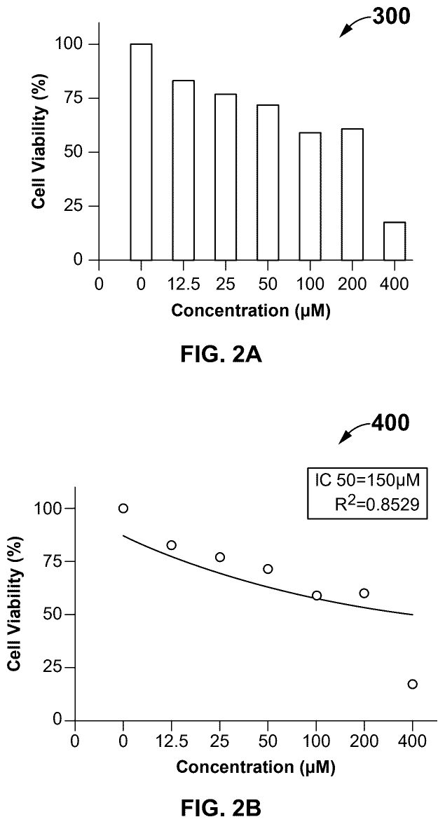

FIG. 2A and FIG. 2B, illustrates the observation of cytotoxicity assay of methyl cinnamate in A549 cells. FIG. 2A illustrates a graph 300 of cytotoxicity of methyl cinnamate in A549 cells is determined based on the dose-response using MTT assay and the 50% cytotoxic concentration (IC₅₀) was calculated for the compound using nonlinear regression analysis of GraphPad Prism software (version 8.0). FIG. 2B is a graph 400 showing the cytotoxicity of methyl cinnamate in A549 cells. The cells were exposed to different concentrations of methyl cinnamate for 48 hours. Values are the mean±SE of three independent experiments.

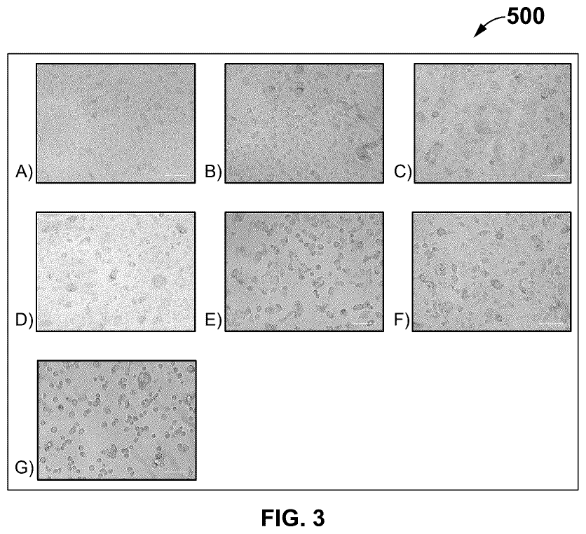

FIG. 3 illustrates microscopic white light images 500 showing toxicity in A549 cell line after 48 hours of treatment with methyl cinnamate at different concentrations. A) at 0 μm (control) concentration, B) at 12.5 μm concentration, C) at 25 μm concentration. D) at 50 μm concentration, E) at 100 μm concentration, F) at 200 μm concentration, and G) at 400 μm concentration. At none and lower concentrations of methyl cinnamate (A, B, C and D) are showing none to very less toxicity, whereas in concentrations above 50 μm (i.e., E, F, and G) circularization of the cells denotes toxicity due to the compound.

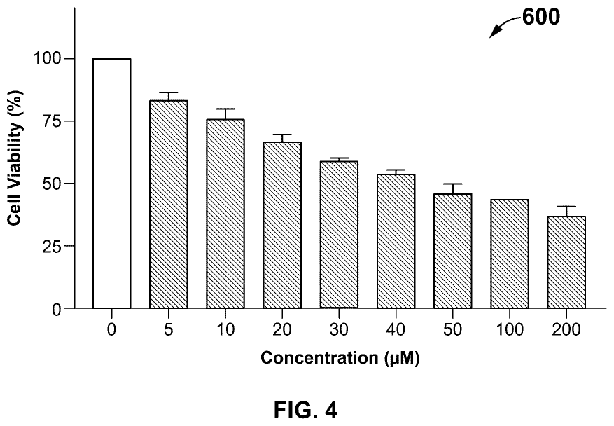

FIG. 4 is a graph 600 showing the cytotoxicity of epicurzerenone in A549 cells. The cytotoxicity of epicurzerenone is determined based on the dose-response using MTT assay. The 50% cytotoxic concentration (IC₅₀) was calculated for the compound using nonlinear regression analysis of GraphPad Prism software (version 8.0). Values are the mean±SE of three independent experiments. Further, the IC₅₀ value of citral, methyl cinnamate, and epicurzerenone based on the observation of cytotoxicity in A549 cells is provided in table 1.

TABLE 1

| S. No. | Cytotoxicity in A549 cells | IC₅₀ |
|---|---|---|
| 1. | Citral | 48 μM |
| 2. | Methyl cinnamate | 150 μM |
| 3. | Epicurzerenone | 35 μM |

Further, the experiment is conducted to check anti-inflammatory response of individual active components. The different concentrations of citral, methyl cinnamate and epicurzerenone is selected based on IC 50 values and used to assess best ant-inflammatory response. The ant-inflammatory response shows good results using the concentration at 2.5 μM of citral, 10 μM of methyl cinnamate and 3 μM of epicurzerenone. Further, the anti-inflammatory effect of individual active components is discussed in FIG. 7 to FIG. 9.

Further, the ribonucleic acid (RNA) is isolated from essential oil treated A549 cells and untreated A549 cells to check the quality and purity of the RNA. The gene expression of inflammatory markers is assessed by an SYBR green-based RT-qPCR. Firstly, RNA is extracted using RNAiso plus (Takara, Japan) reagent as per manufacturer instructions. The RNA is reverse-transcribed into cDNA using High-capacity cDNA Reverse transcription kit (applied biosystem, US). The test gene expression SYBR PCR Master Mix (Applied Biosystems) is conducted with the target genes TNF-α, IL-2, IL-6, and IL-10 with endogenous gene β-actin. The reaction mixture was set up with 5 μl of mastermix (Applied Biosystem, US), 100 ng of cDNA, 100 nM to 500 nM primer mix, and volume makeup with nuclease-free water till 10 μl, with the following thermal conditions: 50° C. for 2 min, 95° C. for 10 min and 40 cycles of 95° C. for 15 sec and 60° C. for 1 min, 95° C. for 15 sec, 60° C. for 1 min. Further, the positive control and No Template Control (NTC) in duplicates were included to rule out any false positive.

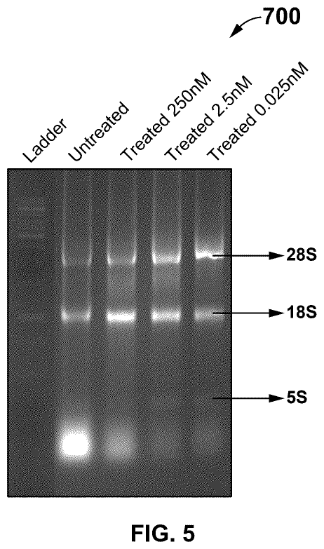

FIG. 5 is a schematic 700 showing Agarose gel (1%) electrophoresis of the total RNA isolated, according to an embodiment of the present invention. The agarose gel has one or more wells. First well of agarose gel is loaded with ladder. The ladder composed of different lengths of DNA molecules that is used to determine the size of unknown DNA/RNA fragments isolated from essential oil treated A549 cells, and untreated A549 cells. Second well of agarose gel is loaded with RNA isolated from untreated A549 cells. Further, third, fourth, and fifth well is loaded with RNA isolated from treated A549 cells. Further, the wells of agarose gel are loaded with ladder, RNA isolated from untreated A549 cells and RNA isolated from untreated A549 cells is incubated for 48 hours. Further, the presence of three intact bands (28S, 18S, and 5S) of RNA is observed. The three intact bands indicate high quality and purity of the RNA isolated for the experiments.

The anti-inflammatory effect of individual active components and binary blends of active components are determined upon post-treatment with LPS induced A549 cells. The Lipopolysaccharide (LPS) induced A549 cells have revealed an upregulation of pro inflammatory cytokines compared to healthy controls. Further, cell mediated immunity is preferentially activated by pro inflammatory cytokines. The pro-inflammatory cytokines include TNF-α and type 1 cytokines that includes IL-2, IL-8, IL-12, IL-15, and interferon (IFN). Further, the preclinical data from A549 cells co-cultures have indicated that overproduction of pro inflammatory cytokines IL-6 and IL-8 during infections might emanate from interaction between bacterial pathogens, neutrophils, and tumor cells which amplify cytokine release and might promote tumor growth.

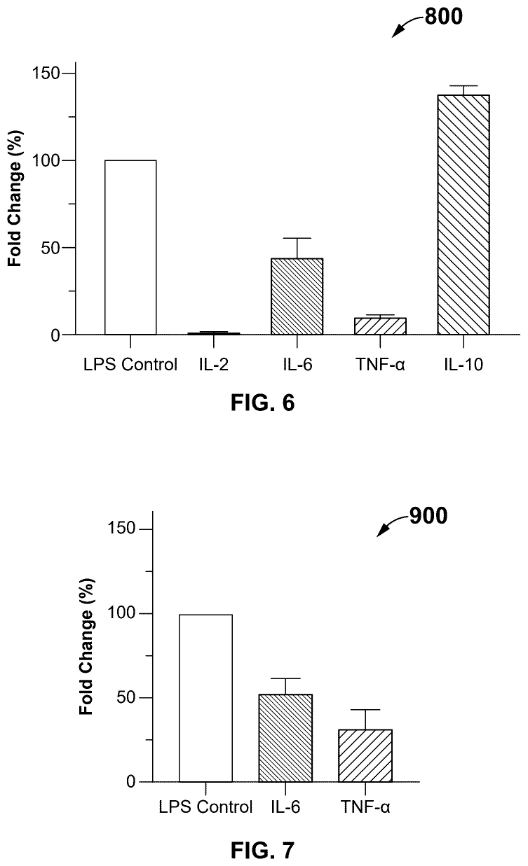

Referring to FIG. 6 to FIG. 8, the anti-inflammatory effect of individual active components is determined upon post-treatment with LPS induced A549 cells. Referring to FIG. 6, the graph 800 illustrates the anti-inflammatory effect of citral upon post-treatment with Lipopolysaccharide (LPS) induced A549 cells for 48 hours. The Modulation of interleukin-2 (IL-2), interleukin-6 (IL-6), interleukin-10 (IL-10) and tumor necrosis factor-alpha (TNF-α) is observed in LPS induced A549 cells after 48 hours of treatment with 2.5 μM of citral. The error bars represent standard deviation from 3 sets of experiments. Further, the graph 800 depicts that pro-inflammatory cytokine includes IL-2, IL-6 and TNF-α are downregulated as compared to the LPS control. Whereas the anti-inflammatory cytokine that include IL-10 is upregulated as compared to the LPS control upon the treatment of citral at a non-toxic level of 2.5 M.

Referring to FIG. 7, graph 900 illustrates the anti-inflammatory effect of methyl cinnamate upon post-treatment with LPS induced A549 cells for 48 hours. The Modulation of interleukin-6 (IL-6), and tumor necrosis factor-alpha (TNF-α) is observed in LPS induced A549 cells after 48 hours of treatment with 10 μM of methyl cinnamate. The error bars represent standard deviation from 3 sets of experiments. Further, the graph 900 depicts that pro-inflammatory cytokines that includes IL-6 and TNF-α are downregulated as compared to the LPS control upon the treatment of methyl cinnamate at a non-toxic level of 10 μM. The modulation of IL-10 is not observed and therefore excluded in the results.

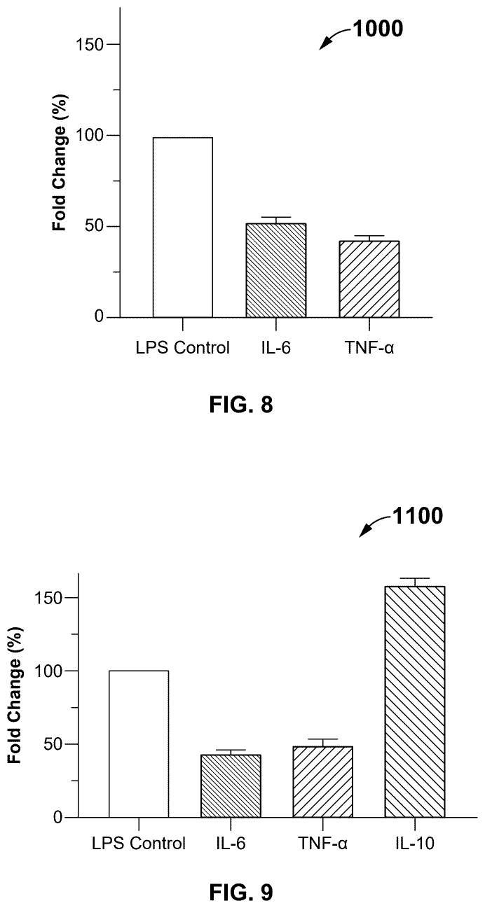

FIG. 8 is a graph 1000 showing an anti-inflammatory effect of epicurzerenone upon post-treatment with LPS induced A549 cells for 48 hours, according to an embodiment of the present invention. The Modulation of interleukin-6 (IL-6), and tumor necrosis factor-alpha (TNF-α) is observed in LPS induced A549 cells after 48 hours of treatment with 3 μM of epicurzerenone. The error bars represent standard deviation from 3 sets of experiments. Further, the graph 1000 depicts that pro-inflammatory cytokines that includes IL-6 and TNF-α are downregulated as compared to the LPS control upon the treatment of epicurzerenone at a non-toxic level of 3 μM. The modulation of IL-10 is not observed and therefore excluded in the results.

The summary of the percent reduction of each of the compounds at their experimental concentration is provided in Table 2.

TABLE 2

| | | | Methyl | |
|---|---|---|---|---|
| | LPS induced | Citral | Cinnamate | Epicurzerenone |
| Cytokines | inflammation | 2.5 μM | 10 μM | 3 μM |
| IL-6 (Pro- | 10 μg/mL | −56% | −49% | −47% |
| inflammatory) | | | | |
| TNF-α (Pro- | 10 μg/mL | −37% | −67% | −56% |
| inflammatory) | | | | |
| IL-10 (Anti- | 10 μg/mL | +39% | N/A | N/A |
| inflammatory) | | | | |

Interpretation: The LPS control is taken at 100% and with respect to the LPS control ‘+’ denotes upregulation and ‘−’ denotes downregulation.

The final concentration is chosen by considering two factors: Firstly, the value needs to be at least 10 times lower than the IC₅₀ value; secondly, the selected concentration needs to show at least 30% of anti-inflammatory response.

Results: For gene expression of single compounds as shown in Table 2, Each compound showed an anti-inflammatory response individually. However, citral showed good results with respect to decreasing pro-inflammatory cytokines and increasing anti-inflammatory cytokines. The citral shows a significantly better anti-inflammatory response compared to epicurzerenone and methyl cinnamate. Further, epicurzerenone shows a significantly better anti-inflammatory response compared to methyl cinnamate. The citral and epicurzerenone is taken comparatively lower concentration compared to methyl cinnamate. Further, the citral and epicurzerenone showed better anti-inflammatory responses.

The anti-inflammatory effect of binary blends of three component are determined upon post-treatment with LPS induced A549 cells. The hypothesis of binary blend combinations of the active compounds might exhibit synergistic effects against inflammation. To test the synergistic effects, three different combinations of the compounds were tried, and gene expression analysis is performed as described in the earlier section.

FIG. 9 is a graph 1100 showing an anti-inflammatory effect of the binary blend comprising 2.5 μM of citral and 10 μM of methyl cinnamate post-treatment with LPS induced A549 cells, according to an embodiment of the present invention. The Modulation of interleukin-6 (IL-6), interleukin-10 (IL-10) and tumor necrosis factor-alpha (TNF-α) is observed in LPS induced A549 cells after 48 hours of treatment with 2.5 μM of citral and 10 μM of methyl cinnamate. The error bars represent standard deviation from 3 sets of experiments. Further, the graph 1100 depicts that pro-inflammatory cytokines that includes IL-6 and TNF-α are downregulated as compared to the LPS control. Whereas the anti-inflammatory cytokine includes IL-10 is upregulated as compared to the LPS control upon the treatment of citral and methyl cinnamate at a non-toxic level of 2.5 μM and 10 μM respectively.

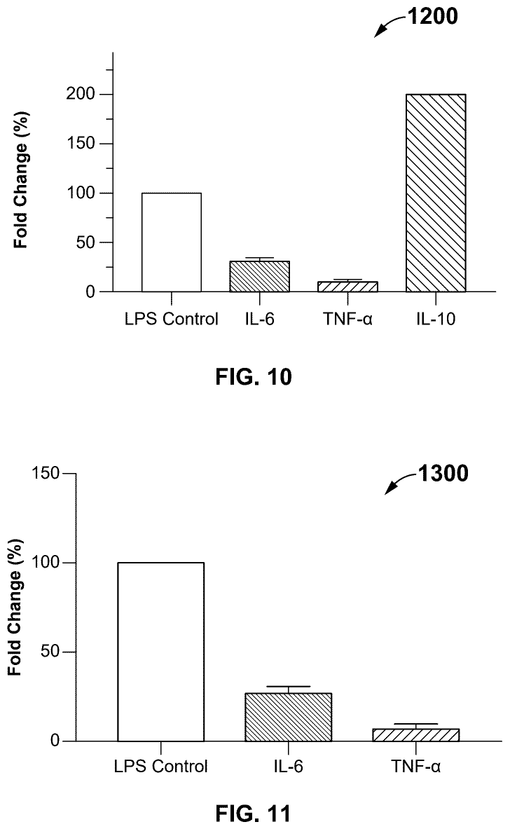

FIG. 10 is a graph 1200 showing an anti-inflammatory effect of the binary blend comprising 10 μM of methyl cinnamate and 3 μM of epicurzerenone post-treatment with LPS induced A549 cells, according to an embodiment of the present invention. The Modulation of interleukin-6 (IL-6), interleukin-10 (IL-10) and tumor necrosis factor-alpha (TNF-α) is observed in LPS induced A549 cells after 48 hours of treatment with 10 μM of methyl cinnamate and 3 μM of epicurzerenone. The error bars represent standard deviation from 3 sets of experiments. Further, the graph 1200 depicts that pro-inflammatory cytokines include IL-6 and TNF-α are downregulated as compared to the LPS control. Whereas the anti-inflammatory cytokine includes IL-10 is upregulated as compared to the LPS control upon the treatment of cinnamate and epicurzerenone at a non-toxic level of 10 μM and 3 μM respectively.

FIG. 11 is a graph 1300 showing an anti-inflammatory effect of the binary blend comprising 2.5 μM of citral and 3 μM of epicurzerenone post-treatment with LPS induced A549 cells. The Modulation of interleukin-6 (IL-6), and tumor necrosis factor-alpha (TNF-α) is observed in LPS induced A549 cells after 48 hours of treatment with 2.5 μM of citral and 3 M of epicurzerenone. Further, the graph 1300 depicts that pro-inflammatory cytokine includes IL-6 and TNF-α are downregulated as compared to the LPS control upon the treatment of citral and epicurzerenone at a non-toxic level of 2.5 μM and 3 M respectively. The modulation of IL-10 is not observed and therefore excluded in the results.

The summary of the percent reduction of each of the binary blend at their experimental concentration is provided in table 3.

TABLE 3

| | LPS | C + MC | C + E | MC + E |
|---|---|---|---|---|
| | induced | 2.5 μM + | 2.5 μM + | 10 μM + |
| Cytokines | inflammation | 10 μM | 3 μM | 3 μM |
| IL-6 (Pro- | 10 μg/mL | −54% | −71% | −70% |
| inflammatory) | | | | |
| TNF-α (Pro- | 10 μg/mL | −50% | −94% | −91% |
| inflammatory) | | | | |
| IL-10 (Anti- | 10 μg/mL | +63% | +100% | N/A |
| inflammatory) | | | | |

Interpretation: The LPS control is taken at 100% and with respect to the LPS control ‘+’ denotes upregulation and ‘−’ denotes downregulation.

The percent reduction of each combination of the binary blends at their experimental concentration. The optimum concentration defined earlier for individual compounds was used for the binary blends of the compounds.

Result: For gene expression of binary blend as shown in Table 3. The binary blend combination shows a superior anti-inflammatory response than single compounds as shown in table 1 and 2. The binary blend combination shows a superior anti-inflammatory response than single compounds as shown in table 1 and 2. The binary blend comprising citral and epicurzerenone (A+C) shows a significantly better anti-inflammatory response compared to binary blend comprising methyl cinnamate and epicurzerenone (B+C), and binary blend comprising citral and methyl cinnamate (A+B). Further, the binary blend comprising methyl cinnamate and epicurzerenone (B+C) shows a significantly better anti-inflammatory response compared to binary blend comprising citral and methyl cinnamate (A+B).

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device, or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A composition for treating arthritis, comprising: a binary blend of molecules including citral, methyl cinnamate, and epicurzerenone, wherein the binary blend exhibits synergistic efficacy in modulating inflammatory cytokines.

2. The composition of claim 1, wherein the citral, methyl cinnamate, and epicurzerenone, are derived from essential oils of Cymbopogon citratus, Hedychium spicatum, and Zanthoxylum armatum, respectively.

3. The composition of claim 1, wherein the binary blend exhibits downregulation of pro-inflammatory cytokines and upregulation of anti-inflammatory cytokines.

4. The composition of claim 1, wherein the binary blend exhibits downregulation of pro-inflammatory cytokines IL-2, IL-6, and TNF-α, and upregulation of anti-inflammatory cytokine IL-10.

5. The composition of claim 1, wherein the binary blend comprises citral of about 2. 5 μM, methyl cinnamate of about 10 μM, and epicurzerenone of about 3 μM.

6. The composition of claim 1, wherein the binary blend is formulated in a pharmaceutically acceptable carrier for at least one of oral, injection and topical administration.

7. The composition of claim 1, wherein the binary blend is administered at doses effective for alleviating symptoms of arthritis in a subject.

8. A method for treating arthritis, comprising steps of: administering to a subject in need thereof, a therapeutically effective amount of a binary blend of molecules comprising citral, methyl cinnamate, and epicurzerenone.

9. The method of claim 8, further comprising a step of: formulating the binary blend in a pharmaceutically acceptable carrier for at least one of oral, injection and topical administration.

10. The method of claim 8, wherein the step of administering the binary blend results in a synergistic efficacy demonstrated by downregulation of pro-inflammatory cytokines and upregulation of anti-inflammatory cytokines compared to individual compounds.

11. The method of claim 8, wherein the step of administering the binary blend comprises selecting concentrations of citral, methyl cinnamate, and epicurzerenone of about 2.5 μM, 10 μM, and 3 μM, respectively.

12. The method of claim 8, wherein the step of administering the binary blend alleviates symptoms of arthritis by downregulating pro-inflammatory cytokines IL-2, IL-6, and TNF-α, and upregulating anti-inflammatory cytokine IL-10, thereby providing therapeutic benefit to the subject.
